(19) United States
(12) Patent Application Publication
(10) Pub. No.: US 2011/0046072 A1
(43) Pub. Date: Feb. 24, 2011

(54) SOLID PHARMACEUTICAL FORMULATION WITH DELAYED RELEASE

(75) Inventors: Venkata-Rangarao Kanikanti, Leverkusen (DE); Thomas Bach, Wuppertal (DE); Michael Traeubel, Koeln (DE); Gertraut Altreuther, Wuppertal (DE); Martina Rehagen, Lohmar (DE); Axel Schmidt, Wuppertal (DE)

Correspondence Address: BAYER HEALTHCARE LLC, P.O.BOX 390, SHAWNEE MISSION, KS, 66201, US

(73) Assignee: BAYER ANIMAL HEALTH GMBH, Leverkusen (DE)

(21) Appl. No.: 12/990,389

(22) PCT Filed: Apr. 23, 2009
(86) PCT No.: PCT/EP09/02951

§ 371 Date: Oct. 29, 2010

(30) Foreign Application Priority Data
May 7, 2008 (DE) 10 2008 022 520.7

(51) Int. Cl. A61K 38/12; A61K 47/32; A61P 1/00; A61P 33/00

(52) U.S. Cl. 514/21.1; 514/772.5

(57) ABSTRACT

The invention relates to a solid pharmaceutical preparation with delayed release of the active ingredients which is suitable in particular for use in animals.

The invention relates to a solid pharmaceutical preparation with delayed release of the active ingredients which is suitable in particular for use in animals.

Pharmaceuticals with delayed release (controlled release formulations, slow-release formulations) are not customary in veterinary medicine, and especially for cats and dogs, although numerous slow-release pharmaceuticals based on various techniques are available for use in humans. One of the main reasons for this is that animals, especially cats and dogs, differ from humans with regard to the transit times in the gastrointestinal tract (gastro-intestinal transit times [GITT]), the influences of food, the influence of the feeding habits, species, size, pH in the stomach, intestinal enzymes, permeability of the gastrointestinal tract and the regions in which active ingredients are absorbed [S. C. Sutton, Adv. Drug delivery reviews. 56 (2004) 1383-1398]. Physiological differences between dogs and humans are described in detail in the literature [Dressman. Pharm Res., 3 (1986) 123-131; Schneider et al., J. Med. Chem., 42 (1999) 5072]. If, on oral intake of a conventional slow-release tablet by an animal, it is chewed or otherwise reduced in size, the active ingredient is released very rapidly and the actual purpose of the slow-release tablet is not achieved. Absorption of the active ingredient or—in other words—the pharmacokinetic profile can be altered considerably by the chewing of the tablet by the animal. The aim is to develop for such cases a formulation with which reduction in size has a minimal influence on the absorption of the active ingredient. The development of such a formulation for use in animals therefore represents a difficult technical problem for the person skilled in the art. It must also be taken into account in this connection that the mechanical stress for example in the canine stomach is considerably greater than in humans.

WO 2004/014346 describes carprofen tablets with delayed release in which the hydrophilic polymers Methocel (hydroxypropylmethylcellulose “HPMC”), Polyox and Carbopol are employed. The tablets are based on the principle that they comprise microparticles which themselves have controlled release properties. Such formulations are complicated to produce, and it is moreover unclear whether a suitable release profile can be achieved in the gastrointestinal tract of the animal without losses of bioavailability.

A further difficulty for the pharmaceutical technologist is that the transit times in the stomach and in the digestive tract may vary considerably. The transit times in the gastrointestinal tract of fasting and fed beagle dogs vary considerably [see, for example, FIGS. 7 and 8 in Sutton. loc. cit.]. Sutton indicates that about 80% of the tablets had a high transit time of more than 24 hours in the digestive tract. He concludes from this that with slow-release formulations having an in vitro release time of less than 24 hours it is normally possible for the complete dose to be absorbed by the dog, and it is not prematurely excreted. The transit time in the stomach also depends on the size of the pharmaceutical and the species and breed of the relevant animal [see, for example, Fix et al., Pharm. Res., 10 (1993) 1087-1089]. The aim is to develop a formulation which is suitable for use in various species and breeds of animals.

A further difficulty for the pharmaceutical technologist derives from the fact that the active ingredient often can be absorbed at all only in certain limited regions of the gastrointestinal tract. If, for example, the active ingredient is absorbed only in the small intestine, the formulation should also release the active ingredient as completely as possible in the small intestine. Variations in the transit time of the formulation in the gastrointestinal tract may influence the bioavailability. A peristaltic movement passes through the digestive tract, e.g. in dogs, at particular time intervals and is also referred to as “housekeeper wave”; this housekeeper wave has an influence on the transit time of the formulation in the gastrointestinal tract, since the transit time depends on whether the housekeeper wave has just started or will be initiated only later. Transit distance times in the stomach also depend considerably on the nature and quantity of the food consumed and even on the size of the pyloric opening.

These difficulties may have contributed to the fact that, to our knowledge, no oral pharmaceuticals with controlled release for dogs are yet on the market. In any event, the development of a formulation with controlled release suitable for example for cats and/or dogs is a difficult task whose solution cannot be inferred directly from the literature.

Numerous possibilities for achieving delayed release are known. The person skilled in the art normally prefers matrix tablets which comprise a polymer such as, for example, cellulose ether (hydroxypropylcellulose or hydroxypropylmethylcellulose) which forms a hydrophilic gel, because such tablets can be produced with machines customary in the pharmaceutical industry and are also insensitive to the production conditions. Even if, for example, a dog chews such a tablet, the fragments swell up because of the gel-forming polymer, and direct rapid release of the active ingredient is delayed thereby.

Animals such as, for example, dogs may vary in size depending on the species and vary in weight. Dosages accurately adapted to the respective size are therefore scarcely available on the market, because this would be much too complicated to produce and sell. This is why the tablets intended for smaller animals are ordinarily also administered to larger animals. In such cases it is necessary to administer two or more tablets to the larger animals. If the customary technique, described above, of hydrophilic matrix tablets which comprise gel-forming polymers such as cellulose ethers is used, the tablets swell in the aqueous medium of the gastrointestinal tract and form a gel envelope. We found in our investigations that the gel layers of such tablets stick together and form large aggregates in the gastrointestinal tract. The surface of such an aggregate is considerably smaller than the total of the surfaces of the individual swollen tablets. This leads to the release rate of the aggregate being substantially lower than that of the individual tablets. The in vivo active ingredient release from the gel matrices is then no longer reproducible.

According to prior art recommendations, a release time of up to 24 hours ought to be acceptable for slow-release tablets for clogs, because the transit time in the gastrointestinal tract (GITT) is at least 24 hours for about >80% of the tablets. However, we unexpectedly found the following: even if cellulose ether-based tablets which release >80% of the active ingredient during about 12 hours in vitro are administered to fasting dogs, tablets which are only partly swollen and have a dry core are found in the faeces. This leads to a reduction in the bioavailability. The problems are further intensified on administration of a plurality of tablets, as is normal practice for larger animals, in order to achieve the correct dosage. In this case, aggregates of adherent tablets are also found in the faeces.

McInnes et al., (Pharm Res., October 2007) investigated two different matrix tablets with different in vitro release rates on fed and fasting dogs. They were unable in their investigations to find a simple correlation between in vitro and in vivo release. This underlines the fact that is not easy to develop a tablet with delayed release which, on the one hand, releases all the active ingredient before excretion with the faeces and which, on the other hand, allows more than one tablet to be administered without disadvantages.

The present invention therefore aims at developing a tablet with delayed release which dissolves as completely as possible in the gastrointestinal tract and releases the active ingredient as completely as possible within a particular time, irrespective of whether the animal has been fed or fasted. This is also important because the animal owner or veterinarian might lose confidence in the product if he finds undissolved parts of the tablet in the faeces. The objective was in particular to find a matrix system which releases preferably at least 80% of all the active ingredient in from 1 to 6 hours and with which no aggregation occurs in the aqueous medium of the gastrointestinal tract if two or more tablets are administered. Finally, it was intended if possible for production to be possible easily with conventional machines of the pharmaceutical industry.

The invention relates to a solid pharmaceutical preparation with delayed release, comprising:

a. at least one pharmaceutically active ingredient
b. polyvinylpyrrolidone with a K value of at least 17
c. at least one filler.

Suitable pharmaceutically active ingredients are in principle all suitable pharmaceutically active chemical compounds.

In a preferred embodiment, these are anthelmintic active ingredients.

A preferred group of anthelmintic active ingredients which may be mentioned are depsipeptides:

Depsipeptides are similar to peptides and differ from the latter in that one or more α-amino acid units are replaced by α-hydroxy carboxylic acid units. Preferably employed according to the invention are cyclic depsipeptides with 18 to 24 ring atoms, in particular with 24 ring atoms.

Depsipeptides with 18 ring atoms include compounds of the general formula (I):

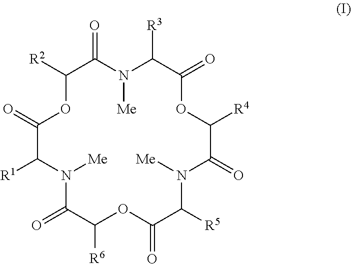

in which
R¹, R³ and R⁵ are independently of one another hydrogen, straight-chain or branched alkyl having up to 8 carbon atoms, hydroxyalkyl, alkanoyloxyalkyl, alkoxyalkyl, aryloxyalkyl, mercaptoalkyl, alkylthioalkyl, alkylsulfinylalkyl, alkylsulfonylalkyl, carboxyalkyl, alkoxycarbonylalkyl, arylalkoxycarbonylalkyl, carbamoylalkyl, aminoalkyl, alkylaminoalkyl, dialkylaminoalkyl, guanidino-alkyl, which may optionally be substituted by one or two benzyloxycarbonyl radicals or by one, two, three or four alkyl radicals, alkoxycarbonylaminoalkyl, 9-fluorenylmethoxycarbonyl(Fmoc)-aminoalkyl, alkenyl, cycloalkyl, cycloalkylalkyl and optionally substituted arylalkyl, where halogen, hydroxy, alkyl and alkoxy may be mentioned as substituents,
R², R⁴ and R⁶ are independently of one another hydrogen, straight-chain or branched alkyl having up to 8 carbon atoms, hydroxyalkyl, mercaptoalkyl, alkanoyloxyalkyl, alkoxyalkyl, aryloxyalkyl, alkylthioalkyl, alkylsulfinylalkyl, alkylsulfonylalkyl, carboxyalkyl, alkoxycarbonylalkyl, arylalkoxycarbonylalkyl, carbamoylalkyl, aminoalkyl, alkylaminoalkyl, dialkylaminoalkyl, alkoxycarbonylaminoalkyl, alkenyl, cycloalkyl, cycloalkylalkyl, optionally substituted aryl or arylalkyl, where halogen, hydroxy, alkyl, alkoxy may be mentioned as substituents, and the optical isomers and racemates thereof.

Preference is given to compounds of the formula (I)

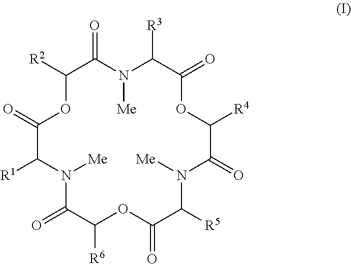

in which
R¹, R³ and R⁵ are independently of one another straight-chain or branched C₁-C₈-alkyl, in particular methyl, ethyl, propyl, isopropyl, butyl, isobutyl, sec-butyl, tert-butyl, pentyl, isopentyl, sec-pentyl, hexyl, isohexyl, sec-hexyl, heptyl, isoheptyl, sec-heptyl, tert-heptyl, octyl, isooctyl, sec-octyl, hydroxy-C₁-C₆-alkyl, in particular hydroxymethyl, 1-hydroxyethyl, C₁-C₄-alkanoyloxy-C₁-C₆-alkyl, in particular acetoxymethyl, 1-acetoxyethyl, C₁-C₄-alkoxy-C₁-C₆-alkyl, in particular methoxymethyl, 1-methoxyethyl, aryl-C₁-C₄-alkyloxy-C₁-C₆-alkyl, in particular benzyloxymethyl, 1-benzyloxyethyl, mercapto-C₁-C₆-alkyl, in particular mercaptomethyl, C₁-C₄-alkylthio-C₁-C₆-alkyl, in particular methylthioethyl, C₁-C₄-alkylsulfinyl-C₁-C₆-alkyl, in particular methylsulfinylethyl, C₁-C₄-alkylsulfonyl-C₁-C₆-alkyl, in particular methylsulfonylethyl, carboxy-C₁-C₆-alkyl, in particular carboxymethyl, carboxyethyl. C₁-C₄-alkoxycarbonyl-C₁-C₆-alkyl, in particular methoxycarbonylmethyl, ethoxycarbonylethyl, C₁-C₄-arylalkoxycarbonyl-C₁-C₆-alkyl, in particular benzyloxycarbonylmethyl, carbamoyl-C₁-C₆-alkyl, in particular carbamoyl methyl, carbamoylethyl, amino-C₁-C₆-alkyl, in particular aminopropyl, aminobutyl. C₁-C₄-alkylamino-C₁-C₆-alkyl, in particular methylaminopropyl, methylaminobutyl, C₁-C₄-dialkylamino-C₁-C₆-alkyl, in particular dimethylaminopropyl, dimethylaminobutyl, guanido-C₁-C₆-alkyl, in particular guanidopropyl, C₁-C₄-alkoxycarbonylamino-C₁-C₆-alkyl, in particular tert-butoxycarbonylaminopropyl, tert-butoxycarbonylaminobutyl, 9-fluorenylmethoxycarbonyl(Fmoc)amino-C₁-C₆-alkyl, in particular 9-fluorenylmethoxycarbonyl(Fmoc)aminopropyl, 9-fluorenylmethoxycarbonyl(Fmoc)aminobutyl, C₂-C₈ alkenyl, in particular vinyl, allyl, butenyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl-C₁-C₄-alkyl, in particular phenylmethyl which may optionally be substituted by radicals from the series halogen, in particular fluorine, chlorine, bromine or iodine, hydroxy, C₁-C₄-alkoxy, in particular methoxy or ethoxy, C₁-C₄-alkyl, in particular methyl,
R², R⁴ and R⁶ are independently of one another straight-chain or branched C₁-C₈-alkyl, in particular methyl, ethyl, propyl, isopropyl, butyl, isobutyl, sec-butyl, tert-butyl, pentyl, isopentyl, sec-pentyl, hexyl, isohexyl, sec-hexyl, heptyl, isoheptyl, sec-heptyl, tert-heptyl, octyl, isooctyl, sec-octyl, hydroxy-C₁-C₆-alkyl, in particular hydroxymethyl, 1-hydroxyethyl, C₁-C₄-alkanoyloxy-C₁-C₆-alkyl, in particular acetoxymethyl, 1-acetoxyethyl, C₁-C₄-alkoxy-C₁-C₆-alkyl, in particular methoxymethyl, 1-methoxyethyl, aryl-C₁-C₄-alkyloxy-C₁-C₆-alkyl, in particular benzyloxymethyl, 1-benzyloxyethyl, mercapto-C₁-C₆-alkyl, in particular mercaptomethyl, C₁-C₄-alkylthio-C₁-C₆-alkyl, in particular methylthioethyl, C₁-C₄-alkylsulfinyl-C₁-C₆-alkyl, in particular methylsulfinylethyl, C₁-C₄-alkylsulfonyl-C₁-C₆-alkyl, in particular methylsulfonylethyl, carboxy-C₁-C₆-alkyl, in particular carboxymethyl, carboxyethyl, C₁-C₄-alkoxycarbonyl-C₁-C₆-alkyl, in particular methoxycarbonylmethyl, ethoxycarbonylethyl, C₁-C₄-arylalkoxycarbonyl-C₁-C₆-alkyl, in particular benzyloxycarbonylmethyl, carbamoyl-C₁-C₆-alkyl, in particular carbamoyl methyl, carbamoylethyl, amino-C₁-C₆-alkyl, in particular aminopropyl, aminobutyl, C₁-C₄-alkylamino-C₁-C₆-alkyl, in particular methylaminopropyl, methylaminobutyl, C₁-C₄-dialkylamino-C₁-C₆-alkyl, in particular dimethylaminopropyl, dimethylaminobutyl, C₂-C₈-alkenyl, in particular vinyl, allyl, butenyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl, phenyl-C₁-C₄-alkyl, in particular phenylmethyl which may optionally be substituted by radicals from the series halogen, in particular fluorine, chlorine, bromine or iodine, hydroxy, C₁-C₄-alkoxy, in particular methoxy or ethoxy, C₁-C₄-alkyl, in particular methyl, and the optical isomers and racemates thereof.

Particular preference is given to compounds of the formula (I) in which

R¹, R³ and R⁵ are independently of one, another straight-chain or branched C₁-C₈-alkyl, in particular methyl, ethyl, propyl, isopropyl, butyl, isobutyl, sec-butyl, pentyl, isopentyl, sec-pentyl, hexyl, isohexyl, sec-hexyl, heptyl, isoheptyl, sec-heptyl, octyl, isooctyl, sec-octyl, hydroxy-C₁-C₆-alkyl, in particular hydroxymethyl, 1-hydroxyethyl, C₁-C₄-alkanoyloxy-C₁-C₆-alkyl, in particular acetoxymethyl, 1-acetoxyethyl, C₁-C₄-alkoxy-C₁-C₆-alkyl, in particular methoxymethyl, 1-methoxyethyl, aryl-C₁-C₄-alkyloxy-C₁-C₆-alkyl, in particular benzyloxymethyl, 1-benzyloxyethyl, C₁-C₄-alkoxycarbonylamino-C₁-C₆-alkyl, in particular tert-butoxycarbonylaminopropyl, tert-butoxycarbonylaminobutyl, C₂-C₈-alkenyl, in particular vinyl, allyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl-C₁-C₄-alkyl, in particular phenylmethyl which may optionally be substituted by one or more identical or different radicals of those indicated above,
R², R⁴ and R⁶ are independently of one another straight-chain or branched C₁-C₈-alkyl, in particular methyl, ethyl, propyl, isopropyl, butyl, isobutyl, sec-butyl, tert-butyl, pentyl, isopentyl, sec-pentyl, hexyl, isohexyl, sec-hexyl, heptyl, isoheptyl, sec-heptyl, tert-heptyl, octyl, isooctyl, sec-octyl, hydroxy-C₁-C₆-alkyl, in particular hydroxymethyl, aryl-C₁-C₄-alkyloxy-C₁-C₆-alkyl, in particular benzyloxymethyl, 1-benzyloxyethyl, carboxy-C₁-C₆-alkyl, in particular carboxymethyl, carboxyethyl, C₁-C₄-alkoxycarbonyl-C₁-C₆-alkyl, in particular methoxycarbonylmethyl, ethoxycarbonylethyl, C₁-C₄-arylalkoxycarbonyl-C₁-C₆-alkyl, in particular benzyloxycarbonylmethyl, C₁-C₄-alkylamino-C₁-C₆-alkyl, in particular methylaminopropyl, methylaminobutyl, C₁-C₄-dialkylamino-C₁-C₆-alkyl, in particular dimethylaminopropyl, dimethylaminobutyl, C₂-C₈-alkenyl, in particular vinyl, allyl, butenyl, C₃-C₇-cycloalkyl, in particular cyclopentyl, cyclohexyl, cycloheptyl, C₃-C₇-cycloalkyl-C₁-C₄-alkyl, in particular cyclopentylmethyl, cyclohexylmethyl, cycloheptylmethyl, phenyl, phenyl-C₁-C₄-alkyl, in particular phenylmethyl which may optionally be substituted by one or more identical or different radicals of those indicated above, and the optical isomers and racemates thereof.

Very particular preference is given to compounds of the formula (I) in which

R¹, R³ and R⁵ are independently of one another straight-chain or branched C₁-C₈-alkyl, in particular methyl, ethyl, propyl, isopropyl, butyl, isobutyl, sec-butyl, pentyl, isopentyl, sec-pentyl, hexyl, isohexyl, sec-hexyl, heptyl, isoheptyl, sec-heptyl, octyl, isooctyl, sec-octyl, C₁-C₈-alkenyl, in particular allyl, C₃-C₇-cycloalkyl-C₁-C₄-alkyl, in particular cyclohexylmethyl, phenyl-C₁-C₄-alkyl, in particular phenylmethyl,
R², R⁴ and R⁶ are independently of one another straight-chain or branched C₁-C₈-alkyl, in particular methyl, ethyl, propyl, isopropyl, butyl, isobutyl, sec-butyl, pentyl, isopentyl, sec-pentyl, hexyl, isohexyl, sec-hexyl, heptyl, isoheptyl, sec-heptyl, octyl, isooctyl, sec-octyl, C₂-C₈-alkenyl, in particular vinyl, allyl, C₃-C₇-cycloalkyl-C₁-C₄-alkyl, in particular cyclohexylmethyl, phenyl-C₁-C₄-alkyl, in particular phenylmethyl which may optionally be substituted by one or more identical or different radicals of those indicated above, and the optical isomers and racemates thereof.

Specific mention may be made of the following compounds of the general formula (I) in which the radicals R¹ to R⁶ have the following meaning:

(I)

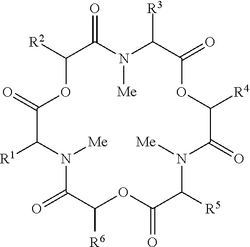

| R¹ | R² | R³ | R⁴ | R⁵ | R⁶ |
|---|---|---|---|---|---|
| —CHMeCH₂Me | -Cyclohexyl | —CHMeCH₂Me | —Me | —CHMeCH₂Me | —Me |
| —CHMeCH₂Me | -Cyclohexyl | —CHMeCH₂Me | —Me | —CHMeCH₂Me | -Cyclohexyl |
| —CHMeCH₂Me | —CH₂-Phe | —CHMeCH₂Me | —Me | —CHMeCH₂Me | —Me |
| —CHMeCH₂Me | —CH₂-Phe | —CHMeCH₂Me | —Me | —CHMeCH₂Me | —CH₂-Phe |
| —CHMeCH₂Me | —(CH₂)₃—Me | —CHMeCH₂Me | —Me | —CHMeCH₂Me | —Me |
| —CHMeCH₂Me | —(CH₂)₃—Me | —CHMeCH₂Me | —Me | —CHMeCH₂Me | —(CH₂)₃—Me |
| —CHMe₂ | —CH₂-Phe | —CHMeCH₂Me | —Me | —CHMeCH₂Me | —Me |
| —CH₂-Phe | —CHMe₂ | —CH₂-Phe | —CHMe₂ | —CHMeCH₂Me | —CHMe₂ |
| —CH₂CHMe₂ | —CH₂-Phe | —CH₂CHMe₂ | —Me | —CH₂CHMe₂ | —CH₂-Phe |
| —(CH₂)₃—Me | —Me | —CHMeCH₂Me | —Me | —CHMeCH₂Me | —Me |
| —CHMe₂ | —Me | —CHMe₂ | —Me | —CHMe₂ | —Me |
| —CH₂—Me | —Me | —CH₂—Me | —Me | —CH₂—Me | —Me |
| —(CH₂)₂—Me | —Me | —(CH₂)₂—Me | —Me | —(CH₂)₂—Me | —Me |
| —(CH₂)₃—Me | —Me | —(CH₂)₃—Me | —Me | —(CH₂)₃—Me | —Me |
| —CH₂—CH═CH₂ | —Me | —CH₂—CH═CH₂ | —Me | —(CH₂)—CH═CH₂ | —Me |
| —CHMeCH₂Me | —Me | —CHMeCH₂Me | —Me | —CHMeCH₂Me | —CH₂—Me |
| —CHMeCH₂Me | —Me | —CHMeCH₂Me | —Me | —CHMeCH₂Me | —(CH₂)₂—Me |
| —CHMeCH₂Me | —Me | —CHMeCH₂Me | —Me | —CHMeCH₂Me | —(CH₂)₃—Me |
| —CHMeCH₂Me | —Me | —CHMeCH₂Me | —Me | —CH₂Me | —Me |
| —CHMeCH₂Me | —Me | —CHMeCH₂Me | —Me | —(CH₂)₂—Me | —Me |
| -Cyclohexyl | —Me | -Cyclohexyl | —Me | -Cyclohexyl | —Me |
| —CH₂CHMe₂ | -Cyclohexyl | —CH₂CHMe₂ | —Me | —CH₂CHMe₂ | -Cyclohexyl |
| —CH₂CHMe₂ | -Cyclohexyl | —CH₂CHMe₂ | —Me | —CH₂CHMe₂ | —Me |
| —CHMeCH₂Me | —CHMe₂ | —CHMeCH₂Me | —CHMe₂ | —CHMeCH₂Me | —Me |
| —CH₂-Phe | —Me | —CH₂-Phe | —Me | —CH₂-Phe | —Me |
| -Cyclohexyl | —Me | -Cyclohexyl | —Me | -Cyclohexyl | —Me |
| —CHMe₂ | —CHMe_{2/} | —CHMe | —Me | —CHMe₂ | —Me |
| —CHMe₂ | —CHMe₂ | —CHMe₂ | —CHMe₂ | —CHMe₂ | —Me |
| —CH₂—Me | —CHMe₂ | —CH₂Me | —Me | —CH₂—Me | —Me |
| —CH₂—Me | —CHMe₂ | —CHMe₂ | —CHMe_{2/} | —CH₂—Me | —Me |
| —(CH₂)₂—Me | —CHMe₂ | —(CH₂)₂—Me | —Me | —(CH₂)₂—Me | —Me |
| —(CH₂)₂—Me | —CHMe₂ | —(CH₂)₂—Me | —CHMe₂ | —(CH₂)₂—Me | —Me |
| —(CH₂)₂—Me | —CHMe₂ | —(CH₂)₃—Me | —Me | —(CH₂)₃—Me | —Me |
| —(CH₂)₃—Me | —CHMe₂ | —(CH₂)₃—Me | —CHMe₂ | —(CH₂)₃—Me | —Me |
| —CH₂—CH═CH₂ | —CHMe₂ | —CH₂—CH═CH₂ | —Me | —CH₂—CH═CH₂ | —Me |
| —CH₂—CH═CH₂ | —CHMe₂ | —CH₂—CH═CH₂ | —CHMe₂ | —CH₂—CH═CH₂ | —Me |
| —Me | —Me | —CHMeCH₂Me | —Me | —CH₂—Me | —Me |
| —Me | —Me | —CHMeCH₂Me | —Me | —(CH₂)₃—Me | —Me |

Me = Methyl;
Phe = Phenyl

Mention may furthermore be made of the compound PF 1022 of the following formula (IIa) disclosed in EP 0 382 173 as depsipeptide:

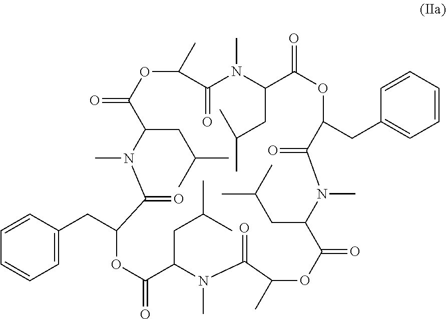

Mention may additionally be made of the compounds disclosed in the PCT application WO 93/19053 as depsipeptides.

Particular mention may be made of the compounds of the following formula (IIb) from WO 93/19053:

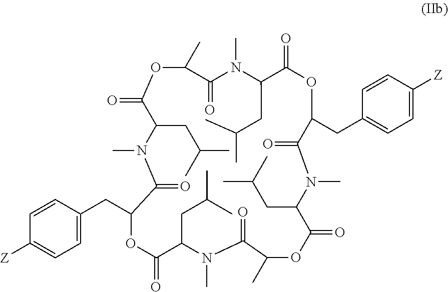

in which
Z is N-morpholinyl, amino, mono- or dimethylamino.

Mention may additionally be made of compounds of the following formula (IIc):

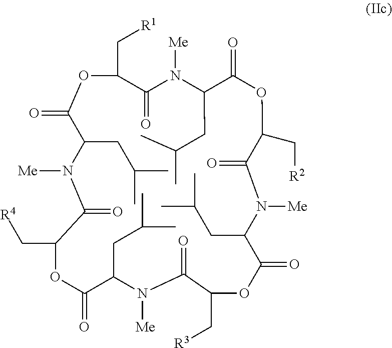

in which
R¹, R², R³, R⁴ are independently of one another hydrogen, C₁-C₁₀-alkyl or aryl, in particular phenyl, which are optionally substituted by hydroxy, C₁-C₁₀-alkoxy or halogen.

The compounds of the general formulae (I) and (IIa), (IIb) and (IIc) are known and can be obtained by the processes described in EP-A-382 173, DE-A 4 317 432, DE-A 4 317 457, DE-A 4 317 458, EP-A-634 408, EP-A-718 293, EP-A-872 481, EP-A-685 469, EP-A-626 375. EP-A-664 297, EP-A-669 343, EP-A-787 141, EP-A-865 498, EP-A-903 347, or in analogy thereto.

Cyclic depsipeptides having 24 ring atoms also include compounds of the general formula (IId)

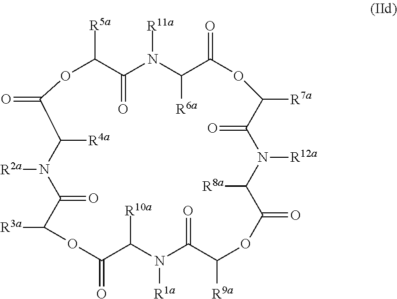

in which
R^{1a}, R^{2a}, R^{11a} and R^{12a} are independently of one another C₁₋₈-alkyl, C₁₋₈-haloalkyl, C₃₋₆-cycloalkyl, aralkyl, aryl,
R^{3a}, R^{5a}, R^{7a}, R^{9a} are independently of one another hydrogen or straight-chain or branched C₁₋₈-alkyl which may optionally be substituted by hydroxy, C₁₋₄-alkoxy, carboxy,

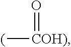

carboxamide,

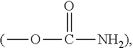

imidazolyl, indolyl, guanidino, SH or C₁₋₄-alkylthio, and is furthermore aryl or aralkyl which may be substituted by halogen, hydroxy, C₁₋₄-alkyl, C₁₋₄-alkoxy,
R^{4a}, R^{6a}, R^{8a}, R^{10a} are independently of one another hydrogen, straight-chain C₁₋₅-alkyl, C₂₋₆-alkenyl, C₃₋₇-cycloalkyl, each of which may optionally be substituted by hydroxy, C₁₋₄-alkoxy, carboxy, carboxamide, imidazolyl, indolyl, guanidino, SH or C₁₋₄-alkylthio, and are aryl or aralkyl which may be substituted by halogen, hydroxy, C₁₋₄-alkoxy, and the optical isomers and racemates thereof.

Preference is given to the use of compounds of the formula (IId) in which

R^{1a}, R^{2a}, R^{11a} and R^{12a} are independently of one another methyl, ethyl, propyl, isopropyl, n-, s-, t-butyl or phenyl which is optionally substituted by halogen, C₁₋₄-alkyl, OH, C₁₋₄-alkoxy, and are benzyl or phenylethyl which may optionally be substituted by the radicals indicated for phenyl;
R^{3a} to R^{10a} have the meaning indicated above.

Particular preference is given to compounds of the formula (IId) in which

R^{1a}, R^{2a}, R^{11a} and R^{12a} are independently of one another methyl, ethyl, propyl, isopropyl or n-, s-, t-butyl,
R^{3a}, R^{5a}, R^{7a} and R^{9a} are hydrogen, straight-chain or branched C₁₋₈-alkyl, in particular methyl, ethyl, propyl, isopropyl, n-, s-, t-butyl, each of which may optionally be substituted by C₁₋₄-alkoxy, in particular methoxy, ethoxy, imidazolyl, indolyl or C₁₋₄-alkylthio, in particular methylthio, ethylthio, and are furthermore phenyl, benzyl or phenethyl, each of which may optionally be substituted by halogen, in particular chlorine,
R^{4a}, R^{6a}, R^{8a} and R^{10a} are independently of one another hydrogen, methyl, ethyl, n-propyl, n-butyl, vinyl, cyclohexyl, each of which may optionally be substituted by methoxy, ethoxy, imidazolyl, indolyl, methylthio, ethylthio, and are isopropyl, s-butyl and furthermore optionally halogen-substituted phenyl, benzyl or phenylethyl.

The compounds of the formula (IId) can likewise be obtained by the processes described in EP-A-382 173, DE-A 4 317 432, DE-A 4 317 457, DE-A 4 317 458, EP-A-634 408, EP-A-718 293, EP-A-872 481, EP-A-685 469, EP-A-626 375, EP-A-664 297, EP-A-669 343, EP-A-787 141, EP-A-865 498, EP-A-903 347.

Depsipeptides which are very particularly preferred according to the invention are PF 1022 A (see formula (IIa)) and emodepside (PF 1022-221, compound of the formula (Mb) in which both Z radicals are the morpholinyl radical). The INN emodepside stands for the compound having the systematic name: cyclo[(R)-lactoyl-N-methyl-L-leucyl-(R)-3-(p-morpholinophenyl)lactoyl-N-methyl-L-leucyl-(R)-lactoyl-N-methyl-L-leucyl-(R)-3-(p-morpholinophenyl)lactoyl-N-methyl-L-leucyl.

Further suitable anthelmintic active ingredients are praziquantel or epsiprantel. Both have long been known as anti-endoparasite active ingredients (see, for example, U.S. Pat. No. 4,661,489 for epsiprantel and U.S. Pat. No. 4,001,411 for praziquantel). Praziquantel-containing products are commercially available, for example under the name Droncit®. The use of praziquantel is preferred in the context of this invention.

In a particularly preferred embodiment, the depsipeptides can be employed in combination with praziquantel or epsiprantel, with praziquantel being preferred as combination partner.

The depsipeptides mentioned above as preferred are also correspondingly preferred or particularly preferred in the combinations.

In a very particularly preferred embodiment, the solid pharmaceutical preparations of the invention comprise a combination of praziquantel and PF 1022 A.

In a further very particularly preferred embodiment, the solid pharmaceutical preparations of the invention comprise a combination of praziquantel and emodepside.

Further suitable active ingredients are macrocyclic lactones, in particular avermectins, dihydroavermectins (ivermectins) or milbemycins. These have an anthelmintic effect but also show a more or less pronounced effect on ectoparasites, for example on insects or mites.

Avermectins in the narrow sense are in particular the eight avermectin components A₁ₐ, A_{1b}, A₂ₐ, A₂ₐ, B₁ₐ, B₂ₐ and B_{2b}. In practice, the mixture referred to as avermectin, which essentially comprises the avermectins B₁, is employed for example. In addition, for example, doramectin and selamectin are included among the avermectins.

The hydrogenation product of abamectin is referred to as ivermectin and is correspondingly 22,23-dihydroavermectins B₁.

Milbemycins which may be mentioned are milbemycin B41 D, nemadectin, moxidectin.

In a further very particularly, preferred embodiment, the solid pharmaceutical preparations of the invention comprise a combination of praziquantel, emodepside and one of the abovementioned macrocyclic lactones. Of these, ivermectin is very particularly preferred in this embodiment.

A further suitable group of active ingredients are analgesics such as, for example, non-opioid analgesics or opioid analgesics. Examples of non-opioid analgesics which may be mentioned are meloxicam, carprofen and metamizole. Examples of opioid analgesics which may be mentioned are buprenorphine and fentanyl.

Metamizole (N-methyl-N-(2,3-dimethyl-5-oxo-1-phenyl-3-pyrazolin-4-yl)aminomethanesulfonic acid, also referred to as dipyrone), which is normally employed in the form of its sodium salt, may be mentioned as a preferred example. Other pharmaceutically acceptable salts can likewise be used. Metamizole is to be regarded more precisely as a prodrug having four main metabolites. Two of these have activity, in particular 4-N-methylaminoantipyrine (4-MAA) and aminoantipyrine (4-AA).

Further active pharmaceutical ingredients which can be employed are pharmacologically acceptable phosphonic acid derivatives, these normally being organic compounds suitable as metabolic stimulants and tonics in particular for productive and domestic animals. Preferred examples which may be mentioned are the compounds, which have been known for a long time, toldimfos and in particular butaphosphan (e.g. used in the product Catosal®), which serve inter alia for mineral (phosphorus) supplementation.

Active ingredients may, depending on the structure, be present in stereoisomeric forms or as mixtures of stereoisomers, e.g. as enantiomers or racemates. Both the mixtures of stereoisomers and the pure stereoisomers can be used according to the invention.

It is further possible to use where appropriate: salts of the active ingredients with pharmaceutically acceptable acids or bases and also solvates, especially hydrates, of the active ingredients or salts thereof.

In a preferred embodiment, the preparations of the invention are tablets.

The preparations comprise release-slowing polymers which are water-swellable polymers. Since the water-swellable polymers form gels in the presence of water, they can also be referred to as “gel-forming polymers”. Examples which may be mentioned are: chitosan, guar gum and polyvinyl acetate. The water-swellable polymers preferably employed according to the invention are polyvinylpyrrolidones or derivatives thereof, but the use of mixtures of polyvinylpyrrolidones and polyvinylpyrrolidone derivatives is also conceivable. However, the use of polyvinylpyrrolidones is particularly preferred.

It is also conceivable to employ polyvinylpyrrolidones in combination with other suitable polymers, mentioning Kollidon® SR from BASF as an example. This comprises a mixture containing spray-dried polyvinyl acetate (with a weight average molecular weight of about 450 000) and soluble polyvinylpyrrolidone (Povidone K 30) in the ratio 8:2.

An example of a suitable polyvinylpyrrolidone derivative which may be mentioned is copovidone (e.g. Kollidon VA 64 from BASF). This is a copolymer of vinylpyrrolidone and vinyl acetate in the ratio 6:4.

Polyvinylpyrrolidones (povidones, PVP) are commercially available hydrophilic polymers suitable for use in solid pharmaceutical preparations with delayed release. Various types of PVP are commercially available. PVP of relatively low molecular weight are normally employed as binders for tablets. PVP swell in aqueous medium and erode. However, it has emerged that PVP-containing tablets do not form a tacky gel layer like, for example, cellulose ethers. According to our in vitro experiments, tablets containing PVP do not adhere even in an aqueous medium. The risk of aggregation in the gastrointestinal tract on administration of a plurality of tablets is low. It is possible by using PVP having different molecular weights to vary the kinetics of release within a defined range.

The polyvinylpyrrolidones or polyvinylpyrrolidone derivatives employed are preferably soluble in water. In this case, the polyvinylpyrrolidones or polyvinylpyrrolidone derivatives are normally linear and not crosslinked.

The polyvinylpyrrolidones or polyvinylpyrrolidone derivatives normally have a K value of at least 17.

The K value of the polyvinylpyrrolidones or polyvinylpyrrolidone derivatives is related to the viscosity and the molecular weight and can be determined by methods known per se. If in doubt, the data on the K value from the European Pharmacopeia (Ph. Eur.) are used.

Preference is given to the use of polyvinylpyrrolidones and/or polyvinylpyrrolidone derivatives with a K value of from 17 to 90, particularly preferably 25 to 90.

The finished formulation normally comprises from 10 to 50% by weight, preferably 15 to 40% by weight, particularly preferably 25 to 35% by weight of polyvinylpyrrolidone or polyvinylpyrrolidone derivative or mixture thereof.

In a preferred embodiment, a polyvinylpyrrolidone and/or polyvinylpyrrolidone derivative with a smaller chain length and one with a greater chain length are employed. The release characteristics can be adjusted particularly well in this way, because a relatively rapid release is achieved with short-chain polyvinylpyrrolidones or polyvinylpyrrolidone derivatives, whereas longer-chain polyvinylpyrrolidones or polyvinylpyrrolidone derivatives lead to slower release. The ratio of longer-chain to shorter-chain polyvinylpyrrolidone or polyvinylpyrrolidone derivative may normally vary in a range from 1:10 parts by weight up to exclusive use of the longer-chain polyvinylpyrrolidone or polyvinylpyrrolidone derivative. The exact ratio should be adjusted according to the diffusion behaviour of the active ingredient used. Active ingredients which are readily soluble in water, such as, for example, metamizole, easily diffuse out of the gel. In this case, suitable kinetics of release can be achieved without short-chain polyvinylpyrrolidone or polyvinylpyrrolidone derivative or with relatively small amounts thereof. The ratio of long-chain to short-chain polyvinylpyrrolidone or polyvinylpyrrolidone derivative is therefore in a preferred embodiment in the range from at least 5:1 by weight up to exclusive use of the long-chain polyvinylpyrrolidone, and the ratio is preferably at least 10:1 by weight.

Active ingredients which are less readily soluble in water, such as emodepside or praziquantel, diffuse out of the gel more slowly and are essentially released on erosion of the gel. A higher proportion of short-chain polyvinylpyrrolidone or polyvinylpyrrolidone derivative is therefore advisable in this case in order to achieve the desired kinetics of release. In a further preferred embodiment, therefore, the ratio of long-chain to short-chain polyvinylpyrrolidone or polyvinylpyrrolidone derivative is in the range from 1:1 to 5:1, preferably 2:1 to 4:1, by weight.

The short-chain polyvinylpyrrolidone or polyvinylpyrrolidone derivative normally has a K value of from 17 to 40, preferably 17 to 30, particularly preferably about 25.

The longer-chain polyvinylpyrrolidone or polyvinylpyrrolidone derivative normally has a K value above 40, preferably 60 to 120, particularly preferably about 90.

Details concerning the abovementioned polyvinylpyrrolidones, polyvinylpyrrolidone derivatives and particular mixtures can be found in the following book: V. Bühler, “Kollidon, Polyvinylpyrrolidone for the pharmaceutical industry,”, 9th revised edition, BASF Pharma Ingredients, Germany, 2008.

It is possible to adjust the release rate of the preparations of the invention by employing water-soluble excipients such as, for example, polyethylene glycol, lactose (especially as lactose monohydrate) or polyhydric alcohols, e.g. mannitol, sorbitol, xylitol or mixtures of the aforementioned excipients; these excipients are present where appropriate in amounts of normally 1 to 20% (m/m), preferably 5 to 15% (m/m).

The release characteristics of the preparations of the invention can be varied further preferably by incorporating disintegrants such as, for example, starch, crosslinked sodium carboxymethyl-cellulose (croscarmellose sodium), sodium starch glycolate, crosslinked polyvinylpyrrolidone (crospovidones, such as, for example, ®Kollidon CL). The use of the aforementioned water-soluble excipients is then not absolutely necessary. A preferred disintegrant is croscarmellose sodium, and a further preferred disintegrant is crospovidone. Where disintegrants are used, they are normally present in amounts of up to 5% (m/m), preferably 0.1 to 3% (m/m), particularly preferably 0.5 to 1.5% (m/m).

It is possible overall by the measures described above to achieve very reproducible bioavailability, and the risk of finding undisintegrated or partly disintegrated tablets in the faeces is very low.

Fillers suitable for solid preparations (e.g. tablets) are customary fillers such as, for example, carbonates such as calcium carbonate, bicarbonates, sodium chloride, aluminium oxides, silicas, aluminas, phosphates (especially calcium phosphates) or organic fillers such as lactose or microcrystalline cellulose. Anhydrous calcium hydrogen phosphate is preferably employed. Microcrystalline cellulose is likewise preferred. It is also possible to combine different fillers together. The total amount of filler(s) is normally 5 to 80% (m/m), preferably 10 to 70% (m/m), particularly preferably 20 to 60% (m/m).

The solid pharmaceutical preparations of the invention may further comprise, besides the active ingredient(s) and the other aforementioned ingredients, also excipients such as, for example: glidants, e.g. colloidal silicon dioxides such as Aerosil®, hydrogenated vegetable oils, stearic acid, talc or mixtures thereof are present where appropriate in amounts of normally 0.1 to 2% (m/m), preferably 0.5 to 1% (m/m). Lubricants such as, for example, magnesium stearate are present where appropriate in amounts of normally 0.3 to 2% (m/m), preferably 0.5 to 1.5% (m/m).

To improve the palatability, in a preferred embodiment aromas and/or flavourings are added.

Suitable as meat aroma are dry liver powders from cattle, poultry, sheep or pigs, preferably from poultry and pigs, and other aroma preparations. In a preferred embodiment, suitable flavourings and aromatizers are mixtures of proteins, fats and carbohydrates which are specially processed; particular mention may be made of Artificial Beef Flavor® from Pharma Chemie (Syracuse, Nebr., USA). Artificial Beef Flavor® is a pig liver extract to which further proteins are added.

In a further preferred embodiment, it is also possible to employ dry liver powders. The flavourings or aromatizers are employed in the pharmaceutical formulations of the invention in an amount of 1-40% by weight, based on the total weight of the finished formulation, preferably 5-30% by weight, in particular 10-25% by weight. The percentage data in this case are percent by weight of the finished formulation.

The preparations of the invention can be produced for example by mixing or granulating the ingredients and then compressing to tablets. Wet granulation processes are preferred. Aromatizers or flavourings, disintegrant, glidant and lubricant are preferably admixed after the granulation, and the mixture is then tableted.

The in vitro release of the preparations of the invention can be determined in conventional release apparatuses, specifically with the paddle test of the US Pharmacopeia (USP) under sink conditions. “Sink conditions” is a term customary in pharmacy and entails the nature and amount of the release medium used being chosen so that three times the amount of the relevant active ingredient would dissolve therein. The maximum volume of release medium is 900 ml. The medium comprises water as essential component, to which a surfactant is added where appropriate to improve the solubility. Conventional buffers are used to adjust the pH in which the relevant active ingredient is most stable. The aim with the formulations of the invention is to achieve at least 80%, preferably at least 85%, in particular at least 90% in vitro release of the active ingredient in 1 to 6 hours, preferably in 1 to 5 hours, particularly preferably in 1.5 to 5 hours. Measurements take place at 37° C. and 75 rpm. In the case of release of depsipeptides, such as emodepside, from depsipeptide-containing formulations, the release medium has a pH of 3.0 (disodium hydrogen phosphate dihydrate/citric acid monohydrate buffer), and 0.5% sodium lauryl sulfate was added. The volume for formulations containing up to 10 mg of emodepside per unit is 500 ml. Sink conditions must be complied with for units (e.g. tablets) with a higher emodepside content. Thus, 900 ml of medium are required for 30 mg units.

In the case of preparations with metamizole, the conditions for determining the in vitro release are as follows: pH 6.8 (phosphate butter, USP standard release medium), 900 ml.

The preparations of the invention are suitable for use in humans and in animal management and animal breeding for productive and breeding livestock, zoo, laboratory, experimental and companion animals.

The productive and breeding livestock include mammals such as, for example, cattle, horses, sheep, pigs, goats, camels, water buffalos, donkeys, rabbits, fallow deer, reindeer, fur-bearing animals such as, for example, mink, chinchilla, raccoon, birds such as, for example, chickens, geese, turkeys, ducks, ostriches.

Laboratory and experimental animals include mice, rats, guinea pigs, golden hamsters, dogs and cats.

Companion animals include dogs and cats. Use in cats and especially dogs is particularly preferred.

In a preferred embodiment, the preparations comprise anthelmintic active ingredients as described hereinbefore. They are then suitable for controlling pathogenic endoparasites which occur in humans and, in animal management and animal breeding for productive and breeding livestock, zoo, laboratory, experimental and companion animals. Depending on the active ingredient employed, they are in this connection effective for all or some stages of development of the pests, and for resistant and normally sensitive types. The intention of controlling the pathogenic parasites is to reduce disease, deaths and reductions in performance (e.g. in the production of meat, milk, wool, hides, eggs, honey etc.) so that the use of the active ingredients makes more economic and simpler livestock management possible. The pathogenic endoparasites include cestodes, trematodes, nematodes, acanthocephales:

from the order of pseudophyllidea for example: Diphyllobothrium spp., Spirometra spp., Schistocephalus spp., Ligula spp. Bothridium spp., Diphlogonoporus spp.
from the order of cyclophyllidea for example: Mesocestoides spp., Anoplocephala spp., Paranoplocephala spp., Moniezia spp., Thysanosomsa spp., Thysaniezia spp., Avitellina spp., Stilesia spp., Cittotaenia spp., Andyra spp., Bertiella spp., Taenia spp., Echinococcus spp. Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.
from the subclass of monogenea for example: Gyrodactylus spp., Dactylogyrus spp., Polystoma spp.
from the subclass of digenea for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp. Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp. Dicrocoelium spp., Eurytrema spp., Troglotrema spp., Paragonimus spp., Collyriclum spp. Nanophyetus spp. Opisthorchis spp., Clonorchis spp. Metorchis spp. Heterophyes spp., Metagonimus spp.
from the order of enoplida for example: Trichuris spp., Capillaria spp., Trichomosoides spp., Trichinella spp.
from the order of rhabditia for example: Micronema spp., Strongyloides spp.
from the order of strongylida for example: Strongylus spp., Triodontophorus spp., Oesophagodontus spp., Trichonema spp., Gyalocephalus spp., Cylindropharynx spp., Poteriostomum spp. Cyclococercus spp., Cylicostephanus spp., Oesophagostomum spp., Chabertia spp. Stephanurus spp. Ancylostoma spp., Uncinaria spp. Bunostomum spp.
Globocephalus spp., Syngamus spp. Cyathostoma spp., Metastrongylus spp. Dictyocaulus spp., Muellerius spp., protostrongylus spp., Neostrongylus spp., Cystocaulus spp., Pneumostrongylus spp., Spicocaulus spp., Elaphostrongylus spp., Parelaphostrongylus spp., Crenosoma spp., Paracrenosoma spp., Angiostrongylus spp., Aelurostrongylus spp., Filaroides spp., Parafilaroides spp. Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp.
from the order of oxyurida for example: Oxyuris spp., Enterobius spp., Passalurus spp., Syphacia spp., Aspiculuris spp., Heterakis spp.
from the order of ascaridia for example: Ascaris spp., Toxascaris spp., Toxocara spp., Parascaris spp., Anisakis spp., Ascaridia spp.
from the order of spirurida for example: Gnathostoma spp., Physaloptera spp., Thelazia spp., Gongylonema spp., Habronema spp. Parabronema spp., Draschia spp., Dracunculus spp.
from the order of filariida for example: Stephanofilaria spp., Parafilaria spp. Setaria spp., Loa spp., Dirofilaria spp., Litomosoides spp., Brugia spp. Wuchereria spp., Onchocerca spp.
from the order of gigantorhynchida for example: Filicollis spp., Moniliformis spp. Macracanthorhynchus spp., Prosthenorchis spp.

The compositions of the invention are particularly preferably used for: Toxocara cati, Toxascaris leonina, Ancylostoma tubaeforme, Dipylidium caninum, Taenia taeniaeformis and Echinococcus multilocularis.

The preparations are also suitable in principle with other active ingredients for the treatment of the indications for which the respective active ingredients are known to be suitable per se.

Analgesics such as metamizole can be employed for example for the treatment of mild and moderate to severe pain, such as, for example: post-traumatic pain (e.g. blunt trauma, distortions), perioperative pain, postoperative pain, tumour pain, osteoarthritic pain, tendopathies, abdominal soft-tissue pain, geriatric toothache.

Both prophylactic and therapeutic use is possible.

### EXAMPLES

### A. Formulation Examples

The following examples are produced by mixing anhydrous calcium hydrogen phosphate, povidone 90 (and, where appropriate, copovidone 64), and a part of the total amount of povidone 25 and microcrystalline cellulose, and then emodepside and praziquantel are mixed in. The mixture is granulated with an aqueous solution of the second part of povidone 25 and dried in a fluidized bed granulator at temperatures below 110° C. The granules are sieved and mixed with Artificial Beef Flavor, sodium croscarmellose, anhydrous colloidal silicon dioxide and magnesium stearate.

The material obtained in this way can be compressed to tablets.

### Example 1

3.00 mg of emodepside
15.00 mg of praziquantel
19.20 mg of anhydrous calcium hydrogen phosphate
37.80 mg of microcrystalline cellulose
31.50 mg of Artificial Beef Flavour (PC 0125, Pharma Chemie Inc., Syracuse/USA)
36.00 mg of povidone 90 (polyvinylpyrrolidone with K value 90)
12.90 mg of povidone 25 (polyvinylpyrrolidone with K value 25)
0.90 mg of anhydrous colloidal silicon dioxide
1.20 mg of crosslinked sodium carboxymethylcellulose (sodium croscarmellose)
1.50 mg of magnesium stearate

### Example 2

10.00 mg of emodepside
50.00 mg of praziquantel
64.00 mg of anhydrous calcium hydrogen phosphate
126.00 mg of microcrystalline cellulose
105.00 mg of Artificial Beef Flavour (PC 0125, Pharma Chemie Inc., Syracuse/USA)
120.00 mg of povidone 90 (polyvinylpyrrolidone with K value 90)
43.00 mg of povidone 25 (polyvinylpyrrolidone with K value 25)
3.00 mg of anhydrous colloidal silicon dioxide
4.00 mg of crosslinked sodium carboxymethylcellulose (sodium croscarmellose)
5.00 mg of magnesium stearate

### Example 3

10.00 mg of emodepside
50.00 mg of praziquantel
64.00 mg of anhydrous calcium hydrogen phosphate
126.00 mg of microcrystalline cellulose
37.50 mg of Artificial Beef Flavour (PC 0125. Pharma Chemie Inc., Syracuse/USA)
40.00 mg of povidone 90 (polyvinylpyrrolidone with K value 90)
42.00 mg of povidone 25 (polyvinylpyrrolidone with K value 25)
2.00 mg of anhydrous colloidal silicon dioxide
4.00 mg of crosslinked sodium carboxymethylcellulose (Sodium Croscarmellose®)
3.75 mg of magnesium stearate

### Example 4

500.00 mg of metamizole sodium
300.00 mg of microcrystalline cellulose
300.00 mg of povidone 90 (polyvinylpyrrolidone with K value 90)
10.00 mg of anhydrous colloidal silicon dioxide
10.00 mg of magnesium stearate

### Example 5

1000.00 mg of metamizole sodium
600.00 mg of microcrystalline cellulose
600.00 mg of povidone 90 (polyvinylpyrrolidone with K value 90)
20.00 mg of anhydrous colloidal silicon dioxide
20.00 mg of magnesium stearate
278.00 mg of dry liver meal

### Example 6

10.00 mg of emodepside
50.00 mg of praziquantel
64.00 mg of anhydrous calcium hydrogen phosphate
126.00 mg of microcrystalline cellulose
105.00 mg of Artificial Beef Flavour (PC 0125, Pharma Chemie Inc., Syracuse/USA)
90.00 mg of povidone 90 (polyvinylpyrrolidone with K value 90)
30.00 mg of copovidone 64 (Kollidon® VA 64 from BASF, copolymer of vinylpyrrolidone and vinyl acetate in the ratio 6:4)
43.00 mg of povidone 25 (polyvinylpyrrolidone with K value 25)
3.00 mg of anhydrous colloidal silicon dioxide
4.00 mg of crosslinked sodium carboxymethylcellulose (sodium croscarmellose)
5.00 mg of magnesium stearate

### Example 7

10.00 mg of emodepside
50.00 mg of praziquantel
64.00 mg of anhydrous calcium hydrogen phosphate
126.00 mg of microcrystalline cellulose
105.00 mg of Artificial Beef Flavour (PC 0125. Pharma Chemie Inc. Syracuse/USA)
30.00 mg of povidone 90 (polyvinylpyrrolidone with K value 90)
90.00 mg of copovidone 64 (Kollidon® VA 64 from BASF, copolymer of vinylpyrrolidone and vinyl acetate in the ratio 6:4)
43.00 mg of povidone 25 (polyvinylpyrrolidone with K value 25)
3.00 mg of anhydrous colloidal silicon dioxide
4.00 mg of crosslinked sodium carboxymethylcellulose (sodium croscarmellose)
5.00 mg of magnesium stearate

### Comparative Example

### Formulation not According to the Invention

5.00 mg of emodepside
50.00 mg of praziquantel
30.00 mg of anhydrous calcium hydrogen phosphate
63.00 mg of microcrystalline cellulose
35.00 mg of Artificial Beef Flavour
92.00 mg of hydroxypropylcellulose M (HPC-M, from Nisso, Japan)
1.00 mg of anhydrous colloidal silicon dioxide
3.00 mg of magnesium stearate

Tablets produced with the formulation of the comparative example reached a release of >80% of the active ingredient within 12 hours in the USP release test. In experiments with dogs, incompletely dissolved tablets and tablet residues were to be found in the faeces. On administration of a plurality of tablets there was adhesion and aggregation.

### In Vitro Release

The in vitro release of the preparations of the invention was determined with the paddle test of the US Pharmacopeia (USP) under sink conditions.

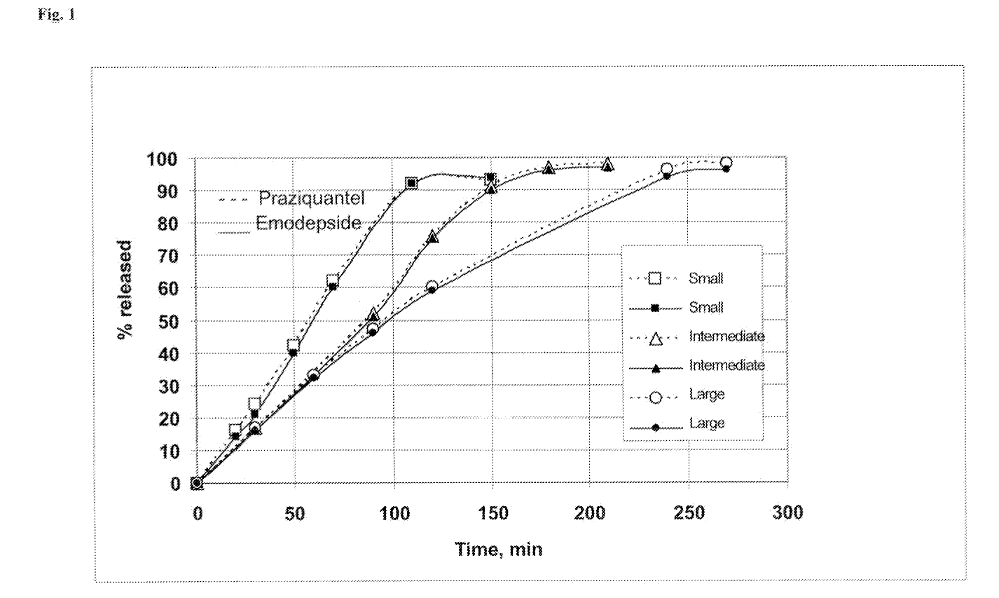

FIG. 1 shows the results for various emodepside/praziquantel tablets:

“Small” stands for the tablets of Example 1
“Intermediate” stands for tablets of Example 2
“Large” stands for larger tablets with 30 mg of emodepside and 150 mg of praziquantel per tablet. The tablets have the same percentage composition as those of Examples 1 and 2.

Measurement conditions: 37° C., 75 rpm, aqueous medium with a pH of 3.0 (disodium hydrogen phosphate dihydrate/citric acid monohydrate buffer), 0.5% sodium lauryl sulfate. 500 ml of release medium were used for the small and intermediate tablets, and 900 ml for the large.

FIG. 1 shows that more than 90% release is reached after 1 to 5 hours with all the tablets.

### A. Biological Examples

### I. Pharmacokinetic Investigations

The medicament to be investigated was administered to fasting dogs. The plasma level of the active ingredient or active ingredients was determined at various times.

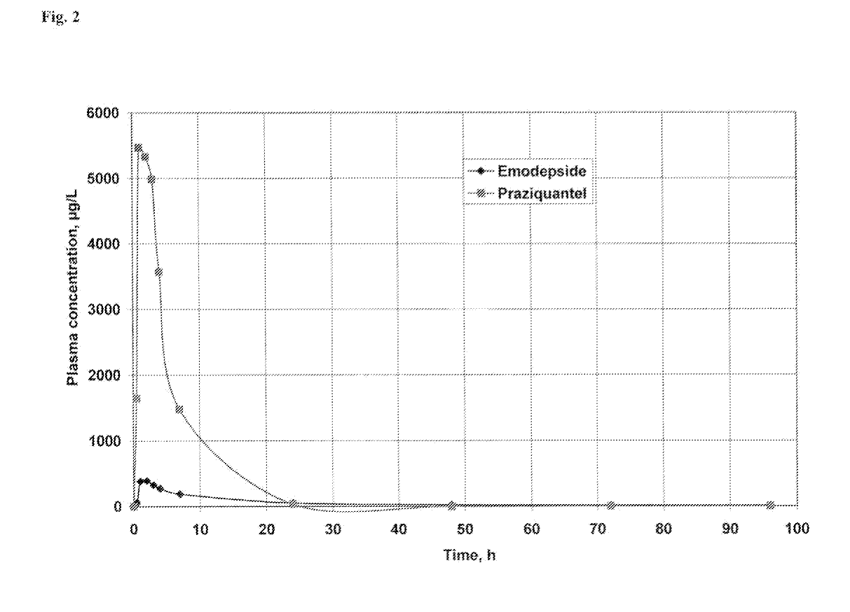

FIG. 2 shows the results after administration of a tablet of Example 2. There is seen to be both with praziquantel and in particular with emodepside to be a distinctly delayed fall in the plasma concentration.

### II. Comparison of a Metamizole Tablet Formulation with a Commercially Available Solution for Intravenous Administration

6 dogs (body weight 9.9-11.1 kg) were divided into two groups each of 3 dogs. One group received intravenous administration of metamizole in the form of the commercially available injection formulation Metapyrin® in a dose of 500 mg/dog. The second group received oral administration of metamizole in the form of the formulation of Example 4, likewise in a dosage of 500 mg/dog. The dogs were fasting when administration took place.

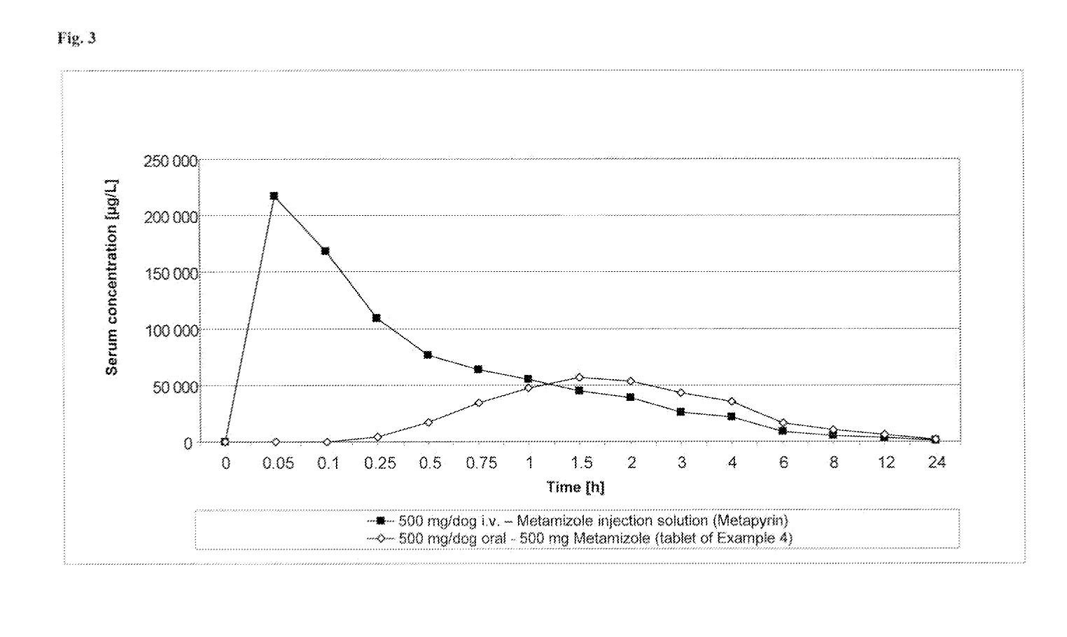

FIG. 3 shows the plasma levels of metamizole after administration, with the metamizole concentration indicated [4-MAA+4-AA] being a calculated value determined from the total of the serum concentrations of the two active main metabolites 4-MAA and 4-AA taking account of the molecular mass of these two metabolites.

### III. Plasma Concentration after Oral Administration of One or Two Metamizole Tablets

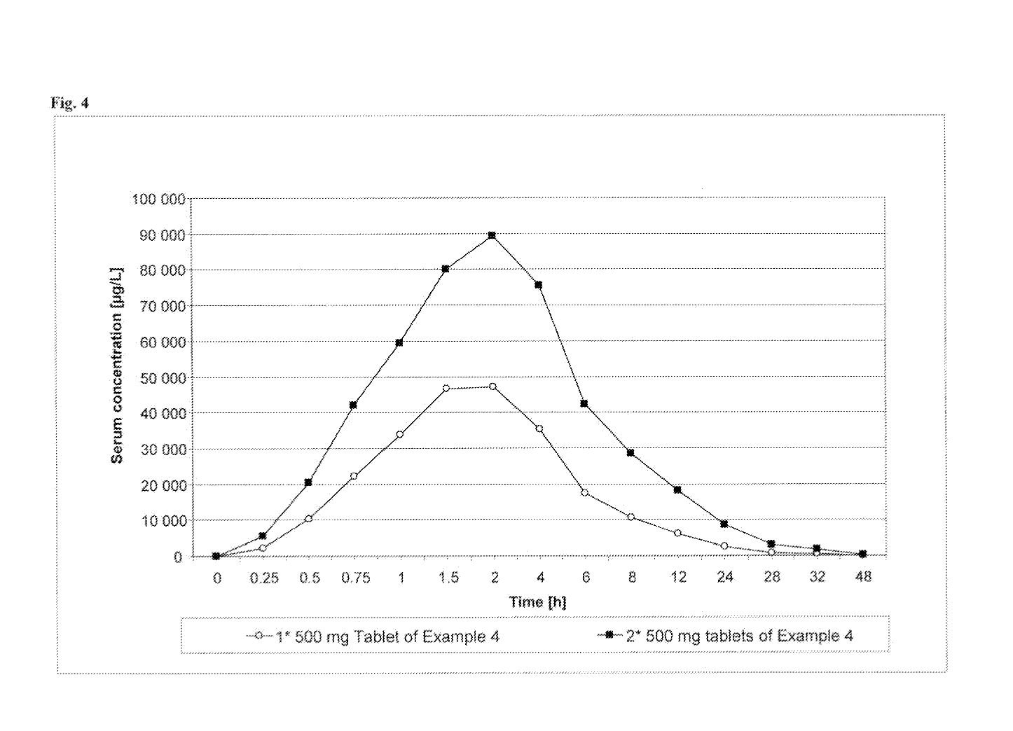

FIG. 4 shows the average metamizole concentration [4-MAA+4-AA] in the serum of fasting dogs after oral administration of one or two metamizole tablets of Example 4. It is evident that the plasma levels correlate very well with the dose administered.

1. Solid pharmaceutical preparation with delayed release, comprising:
a. at least one pharmaceutically active ingredient;
b. polyvinylpyrrolidone and/or a polyvinylpyrrolidone derivative with a K value of at least 17; and,
c. at least one filler.

2. Solid pharmaceutical preparation according to claim 1, comprising from 10 to 50% by weight of polyvinylpyrrolidone.

3. Solid pharmaceutical preparation according to claim 1, additionally comprising a disintegrant.

4. Solid pharmaceutical preparation according to claim 3, comprising the disintegrant in amounts of up to 5% (m/m).

5. Solid pharmaceutical preparation according to claim 1, wherein the active ingredient is a depsipeptide.

6. Solid pharmaceutical preparation according to claim 5, wherein the depsipeptide is emodepside.

7. Solid pharmaceutical preparation according to claim 6, further comprising praziquantel.

8. Solid pharmaceutical preparation according to claims 1, wherein the active ingredient is an analgesic.

9. Solid pharmaceutical preparation according to claim 8, wherein the analgesic is metamizole.

10. Solid pharmaceutical preparation according to claims 1, wherein the active ingredient is a macrocyclic lactone.

11. Solid pharmaceutical preparation according to claim 10, wherein the macrocyclic lactone is ivermectin.

12. Solid pharmaceutical preparation according to claims 1, wherein the active ingredient is a pharmacologically acceptable phosphonic acid derivative.

13. Solid pharmaceutical preparation according to claim 12, wherein the phosphonic acid derivative is butaphosphan.

14. Solid pharmaceutical preparation according to claim 1, characterized in that it releases 80% of the active ingredient in the paddle test of the US Pharmacopeia at 37° C. and 75 revolutions per minute under sink conditions within 1 to 6 hours.
